(19) United States
(12) Patent Application Publication
(10) Pub. No.: US 2007/0112377 A1
(43) Pub. Date: May 17, 2007

(54) Reusable laparoscopic surgical instrument

(76) Inventor: James A. Schneiter, Lake Forest, IL (US)

Correspondence Address: BRINKS HOFER GILSON & LIONE, P.O. BOX 10395, CHICAGO, IL, 60610, US

(21) Appl. No.: 11/593,451

(22) Filed: Nov. 6, 2006

Related U.S. Application Data
(60) Provisional application No. 60/734,827, filed on Nov. 9, 2005.

(51) Int. Cl. A61B 17/00

(52) U.S. Cl. 606/205

(57) ABSTRACT

A surgical instrument having a handle, an elongated housing and a movable rod is provided. The housing has a proximal end and a distal end. The handle and the housing define an interior passageway. The elongated housing has a diameter substantially larger than a diameter of the interior passageway. A movable rod is located within the passageway. A tool is connected to the rod and extends from the distal end of the elongated housing.

### RELATED APPLICATIONS

This patent application claims priority and the benefit under 35 U.S.C. §119(e) to U.S. Provisional Patent Application No. 60/734,827, filed Nov. 9, 2005, which is hereby incorporated by reference.

### FIELD OF THE INVENTION

The present invention relates generally to the field of surgical instruments, such as laparoscopic instruments. More specifically, the present invention relates to a reusable hand held laparoscopic surgical instrument that prevents the infiltration of C02 through the instrument during the surgical procedure while having improved flushing, decontamination, cleaning, sterilization, and post-sterilization moisture elimination capabilities. The present invention also has an improved construction that helps prevent the collection of bio-burden on the outside portion of the instrument and an improved insulating material(s) to reduce the risk of a patient burn.

### BACKGROUND OF THE INVENTION

The use of surgical instruments such as laparoscopic instruments for doing minimally invasive surgical procedures is known. A major consideration with the use of these reusable laparoscopic instruments is the protection of patients' from a deep organ surgical site infection (SSI) caused by residual bio-burden and/or waterborne pathogens that remain inside the lumen of the instrument after reprocessing. “The Centers for Disease Control and Prevention (CDC) reports that hospital acquired infections (HAI's) cause 90,000-plus patients to die of the total two million that contact these infections annually. This mortality figure is more than the combined total number of deaths attributed to AIDS, breast cancer and motor vehicle accidents.” (Thomas L. Kovach, Infection Control Today, June 2005).

Surgical infections account for nearly one-third of all HAI's, or over 667,000 annually in the United States. Approximately one-half of all surgical procedures done in the U.S. are performed laparoscopically and account for over 333,000 surgical infections and over 15,000 patient deaths annually. Any and all steps taken to reduce the risk of an SSI caused by a reusable laparoscopic instrument contaminated by bio-burden and/or waterborne pathogens that remain inside the lumen of the instrument after sterile reprocessing will have an immediate, positive impact on reducing the risk of an SSI and will contribute to improved patient outcomes.

The goal of laparoscopic instrument reprocessing is to ensure a decontaminated, clean, sterile, and moisture-free instrument for every laparoscopic surgical procedure. There are three main elements in instrument reprocessing that must be accomplished every time in order to achieve this goal. First, the instrument must be designed so that the reprocessing technician can remove bio-buren from inside the lumen of the instrument and the outside housing of the instrument prior to the sterilization cycle. Second, the instrument must be designed to ensure 100% bacteria kill on every sterilization cycle. Third, the instrument must be designed to be “moisture free” after every sterilization cycle in order to eliminate the risk of contamination caused by the presence of waterborne pathogens trapped inside the lumen.

An important problem associated with the use of reusable laparoscopic instruments is the removal of the bio-burden and microorganisms that collect inside the lumen of the instrument. During a surgical procedure, the distal end of the instrument is placed inside the patient and the lumen comes into contact with the patient's bio-burden (blood, microorganisms, protein, fat, starches and carbohydrates). In order to provide adequate visualization during a laparoscopic procedure, the patient's abdominal cavity is insufflated with C02 gas to two times the normal atmospheric pressure. During a laparoscopic procedure, some of the pressurized C02 gas escapes through both the luer port on the handle and through the opening for the operating rod in the handle. As the high pressure C02 escapes through the instrument, it pulls some of the patient's bio-burden into the instrument, coating the wall of the lumen and the operating rod. Once inside the lumen of the instrument and on the operating rod, the bio-burden is difficult, if not impossible, to remove during cleaning and decontamination.

In the April 2001 AORN Journal, Romona Conner states, “[d]econtamination is the first and most important step in the sterilization process. Inadequate cleaning of organic debris (bio-burden) may result in retained organisms and make the sterilization process ineffective.” In the June 2002 issue of Infection Control Today, Kelly M. Pyrek states, “[i]nadequate cleaning has the potential to allow for residual bio-burden to be sequestered in bodily fluids that may be contaminated with gram-negative bacteria. You can sterilize it but you may fail to destroy microbial endotoxins that are heat-stable. So cleaning is an absolutely crucial step before any terminal disinfection or sterilization process.”

In the CDC's report Guideline for the Prevention of Surgical Site Infection, 1999 the authors state, “[a]mong surgical patients, SSI's (surgical site infections) were the most common infection, accounting for 38% of all such infections. Of these SSI's, two-thirds were confined to the incision, and one-third involved organs or spaces accessed during the operation. When surgical patients with SSI's died, 77% of the deaths were reported to be related to the infection, and the majority (93%) were serious infections involving organs or spaces accessed during the operation.”

During a surgical laparoscopic procedure, nothing comes in contact more frequently with the organs or spaces accessed during the operation more than the laparoscopic instruments. In the article Infection Control Challenges With Laparoscopic Instruments (Infection Control Today, November 2002) author Ann Hewitt, RN, BSN, MM, states, “[r]eusable laparoscopic instruments that are not (or cannot be) properly cleaned and sterilized are a major cause of deep organ SSI's. The CDC notes ‘Inadequate sterilization of surgical instruments has resulted in SSI outbreaks’ and cites articles in Anesthesiology, MMWR and Journal of Hospital Infections in asserting this claim.” Hewitt goes on to point out that “[d]ue to the design of internal lumens and channels in many laparoscopic instruments, it is impossible to access the entire surface area that needs cleaning. Squared off comers, dead spaces and rough edges all provide nooks and crannies for the deposit of tissue, blood, mucous or other bio-burden. Devices that you know are damaged, corroded, bent or constructed with inaccessible surfaces that come into contact with patient tissue should not be used on patients.”

Another problem caused by the internal design of laparoscopic instruments, both “one-piece” and “take-a-part”, is that it is difficult, if not impossible, to remove residual moisture from inside the lumen of the instrument after the sterilization cycle. Residual moisture has the potential to colonize and grow waterborne pathogens inside the lumen of the instrument. Waterborne pathogens have been documented to be a significant cause of hospital acquired infections (HAI's). Although a skilled technician can remove bio-burden from the lumen of a “take-a-part” laparoscopic instrument, once it has been re-assembled and sterilized, it is very difficult to remove the residual moisture that becomes trapped inside the instrument during sterilization. The presence of waterborne pathogens trapped inside the lumen of an instrument has the potential to cause a deep organ surgical infection, despite the instrument having gone through the cleaning and sterilization process.

Another problem with conventional reusable laparoscopic instruments is that this outer housing or shaft has always been made out of stainless steel with a wall thickness of 1mm or less, resulting in a lot of “dead space” within the lumen of the instrument. This excess “dead space” allows for the collection, retention and build-up of infectious bio-burden within the lumen of the shaft thereby reducing the effectiveness of the detergent flush during cleaning.

A further issue with some reusable laparoscopic instruments is a problem with the insulation on the external surface of the housing or shaft. In particular, the external housing (starting from the handle and running to the opening for the clevis and jaw) has been covered with a “shrink film” type of insulating film (approximately 0.3-0.5 mm in thickness) to protect the patient from dangerous electrical burns. Insulation materials vary, but no matter the source, all conventional shrink film materials are generally degradable with use.

This thin layer of insulation causes two major problems. First, with normal use, it develops “pin holes” and “cracks” that allow electrical current to leak through the compromised insulation and burn the patient's tissue and internal organs surrounding the targeted surgical site. Because the keyhole of minimally invasive surgery is so small, the surgeon cannot observe such phenomena outside his field of vision. Most injuries caused by insulation failure result in irreversible tissue death. Diagnosis is difficult and often delayed. Complications include perforated organs, permanent disfigurement, and in an estimated 28% of fecal peritonitis cases, even death. Accordingly, it is critical to patient safety that insulation be flawless to prevent escaping current. The Association of periOperative Registered Nurses (AORN) Board of Directors in its Recommended Practices for Electrosurgery advises, “[t]he active electrode instrument should be inspected for damage, including impaired insulation, at the operative field before use.” Replacing damaged insulation requires that the instrument be taken out of service and sent to a skilled repair facility.

The second major problem caused by this thin layer of insulation is the gradual collection and retention of infectious bio-burden between the insulation and the stainless steel shaft at the distal end of the instrument. With each reprocessing cycle, the thin insulation is subject to the heat of sterilization (275 degrees F.) thereby causing the stainless steel shaft and insulation to expand slightly. As the instrument cools to room temperature, the stainless steel shaft contracts to its original diameter, however, the “shrink film” insulation does not contract to its original dimension, and thus creating a small gap between the insulation and the shaft. This small gap grows with each reprocessing cycle. As this gap continues to grow in size and in length along the shaft, it serves to collect and retain infectious bio-burden that is difficult, if not impossible to remove during reprocessing and, left in place, can cause a deep organ surgical infection.

Instruments that cannot be flushed and cleaned properly cannot be disinfected or sterilized with certainty, and have been documented to cause life threatening deep organ SSI's. Accordingly, there exists a need for a reusable laparoscopic instrument that prevents the escape of C02 during the surgical procedure and that provides for the efficient flushing, decontamination, cleaning, sterilization, and post-sterilization moisture elimination of the instrument on every reprocessing cycle. There also exists a need for a reusable laparoscopic instrument that has an improved insulation system that solves the problems inherent in conventional “shrink film” insulation systems.

### SUMMARY OF THE INVENTION

The present invention is directed to a surgical instrument that decreases the accessibility of the interior of the instrument to bio-burden while improving the flushing, decontamination, cleaning, sterilization, and post-sterilization moisture elimination of the instrument.

According to a first aspect of the invention, a surgical instrument having a handle, an elongated housing and a movable rod is provided. The housing has a proximal end and a distal end. The handle and the housing define an interior passageway. The elongated housing has a diameter substantially larger than a diameter of the interior passageway. A movable rod is located within the passageway. A tool is connected to the rod and extends from the distal end of the elongated housing.

According to a second aspect of the invention, a surgical instrument having a handle, an elongated housing and a movable rod is provided. The elongated housing is connected to the handle. The housing has a proximal end and a distal end. The housing and the handle define an interior passageway. A movable rod is located within the passageway of the handle and the housing. The rod passes through a rear portion of the handle and through the passageway. A tool is connected to a distal end of the rod. A seal is connected to the rod and handle adjacent the passageway.

According to a third aspect of the invention, a surgical instrument having a handle, an elongated housing and a movable rod is provided. The elongated housing is connected to the handle. The handle and the housing define an interior fluid passageway. A movable rod is located within the fluid passageway of the handle and housing. A port is connected to the handle and is in fluid communication with the interior fluid passageway. A valve is connected to one of the port and the passageway.

According to a fourth aspect of the invention, a surgical instrument having a handle, an elongated housing and a movable rod is provided. The housing has a proximal end and a distal end. The handle and the housing define an interior passageway. The housing is formed from a first nonconductive material. A movable rod is located within the passageway. A tool is connected to the rod and extends from the distal end of the elongated housing.

The present invention, together with attendant objects and advantages, will be best understood with reference to the detailed description below in connection with the attached drawings.

### BRIEF DESCRIPTION OF THE DRAWINGS

FIG. 1 is an illustration of a laparoscopic instrument in accordance with a first embodiment of the present invention;

FIG. 2A is an enlarged view of a proximal end of the instrument of FIG. 1 according to one embodiment;

FIG. 2B is an enlarged view of a proximal end of the instrument of FIG. 1 according to another embodiment illustrating an alternate location of the valve;

FIG. 2C is an enlarged view of a proximal end of the instrument of FIG. 1 according to a further embodiment illustrating an alternate embodiment of the operating rod and lumen;

FIG. 2D is an enlarged view of a proximal end of the instrument of FIG. 1 according to a further embodiment illustrating an alternate embodiment with a cap;

FIG. 2E is a view of one alternate embodiment of the cap shown in FIG. 2D;

FIG. 2F is a view of a second alternate embodiment of the cap shown in FIG. 2D;

FIG. 3 is an enlarged view of the port of FIG. 2A illustrating the location of the valve;

FIG. 4 is an enlarged view of a seal in the proximal end of the instrument as illustrated in FIG. 2A;

FIG. 5A is an enlarged view of a distal end of the instrument according to the embodiment of FIG. 2A illustrating the rod, throat and tool; FIG. 5B is an enlarged view of a distal end of the instrument according to the embodiment of FIG. 2C illustrating the rod, throat and tool; and

FIG. 6 is an illustration of further embodiment of a laparoscopic instrument in accordance with the present invention.

### DETAILED DESCRIPTION OF THE PREFERRED EMBODIMENTS

The present invention is directed to a surgical instrument such as a reusable laparoscopic instrument with a dramatically reduced surface area/volume of the internal lumen or passageway in order to decrease the collection and retention of bio-burden and waterborne pathogens and increase flushing efficiency. The present invention also prevents the escape of C02 from the insufflated abdominal cavity and thereby minimizes the infiltration and collection of bio-burden during the surgical procedure. The present invention also has an internal lumen or passageway that includes a throat portion featuring a “Venturi” type design. The “Venturi” design has advantages including one or more of the following: (a) minimizing the collection of bio-burden during surgery; (b) maximizing the cleaning and flushing efficiency of the instrument during decontamination to remove sequestered bio-burden; (c) maximizing steam penetration throughout the entire internal lumen of the instrument during the sterilization cycle to ensure 100% bacteria kill; and (d) eliminating the collection and retention of moisture both during and after the sterilization process to eliminate the potential for contamination by waterborne pathogens.

In one embodiment, the instrument includes the use of a lumen with dramatically reduced surface area/volume. By using a lumen that defines a relatively small surface area, the opportunity of the collection and retention of bio-burden and waterborne pathogens is decreased. In addition, the small surface area of the lumen provides for an increased flushing efficiency.

In another embodiment, the instrument includes a housing formed out of a nonconducting medical grade material that has improved durability and insulating properties.

The preferred embodiments are described generally in the context of laparoscopic surgical instruments. However, the principles of the invention apply equally well to other types of surgical instruments that have enclosed lumens and come in contact with bio-burden and microorganisms during surgical procedures.

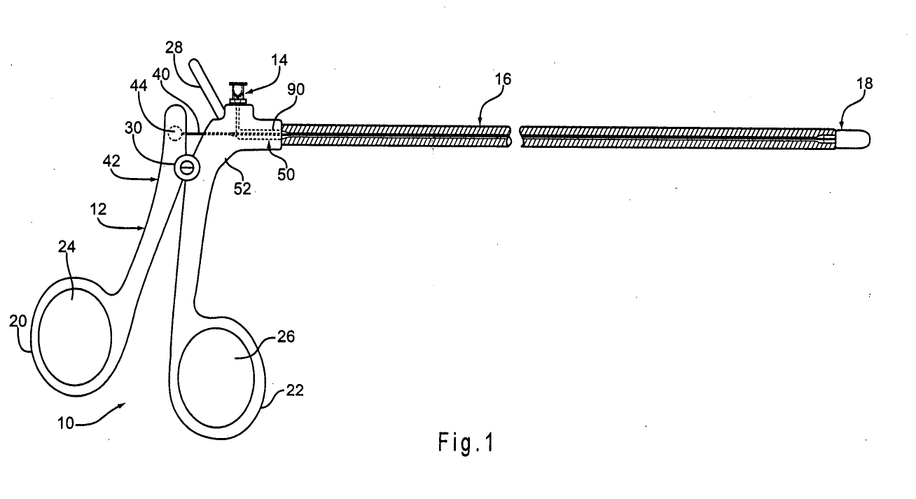

FIG. 1 illustrates a laparoscopic surgical instrument 10 in accordance with a first embodiment of the present invention. The instrument 10 includes a handle 12, a port 14, an elongated housing 16 and a tool 18. The handle 12 includes a thumb portion 20 and a finger portion 22. The thumb portion 20 includes an opening 24 adapted to receive the thumb of a user. The finger portion 22 includes an opening 26 adapted to receive the finger of a user. An electrode post 28 for use in electrosurgery is located on the finger portion 22.

According to one embodiment of the present invention, the elongated housing 16 or shaft may be formed out of a nonconducting medical grade material that has improved durability and insulating properties compared to the use of a conventional “shrink film” insulation covering a stainless steel housing. More specifically, a non-porous, non-ferrous material such as a unidirectional graphite carbon fiber, Radel®, Poly Sulfoam®, Delrin®, high durometer nylon, and various other commercial graphite-fiber composites may be used to construct the housing 16. These materials will help prevent the formation of holes and cracks that could let electric current leak through the exterior of the instrument and thus cause a patient burn. In addition, these materials will help prevent the collection of bio-burden on the exterior of the instrument. Alternatively, the housing 16 could be formed from conventional materials such as stainless steel or titanium and then insulated using conventional shrink film materials.

The thumb portion 20 and the finger portion 22 may be formed from conventional materials such as stainless steel or other metals such as titanium. Other materials such as carbon fiber or plastics such as Delrin™ may also be used. The handle 12 may also include an insulating coating such as nylon or Teflon™ in order to protect the user from electrical current.

A pin 30 interconnects the thumb portion 20 and the finger portion 22 and defines a pivot axis for the handle 12. The thumb portion 20 is movable relative to the finger portion 22. An operating rod 40 is connected to an upper portion 42 of the thumb portion 20 and movable therewith. As used herein, the term “rod” should be interpreted broadly to include structures having various shapes, in the cross-section, such as round, rectangular or triangular. A ball 44 (shown in phantom lines) is welded to an end of the operating rod 40. The ball 44 is located within a pocket within the thumb portion 20 in order to secure the operating rod 40 thereto. In operation, as a user pulls backwards on the thumb portion 20, the operating rod 40 is pushed forward. As a user pushes forward on the thumb portion 20, the operating rod 40 is pulled backward. The operating rod 40 slides within the lumen or passageway 50 formed in an upper portion 52 of the finger portion 22 of the handle 12. The sliding action of the operating rod 40 opens and closes the tool 18.

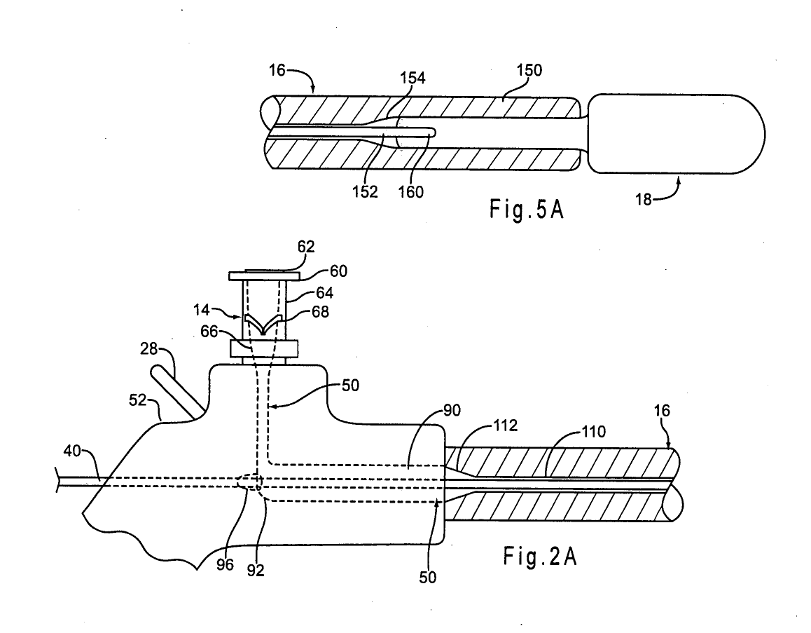

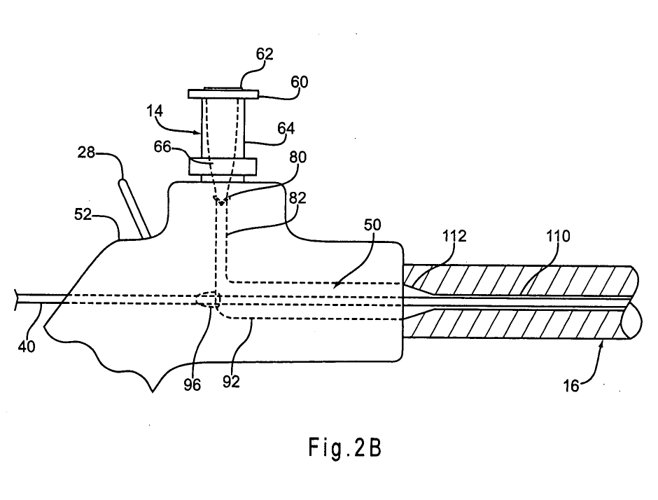

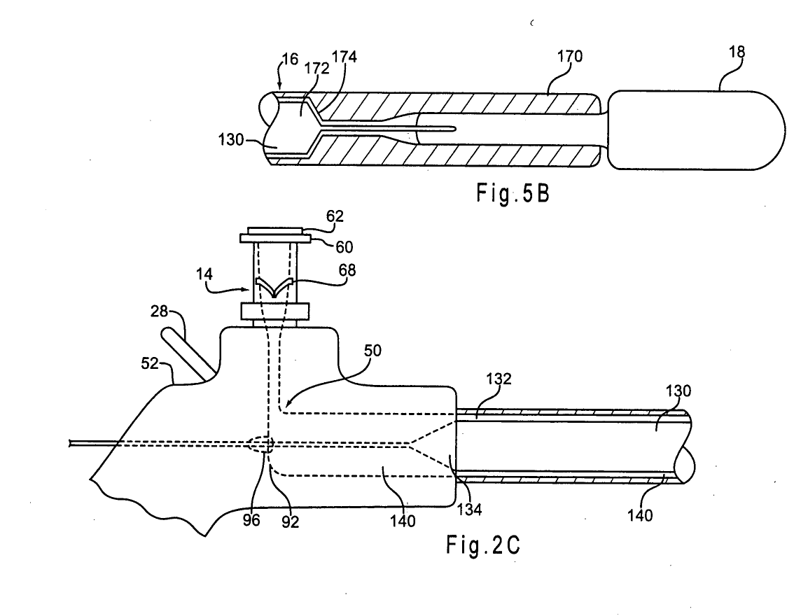

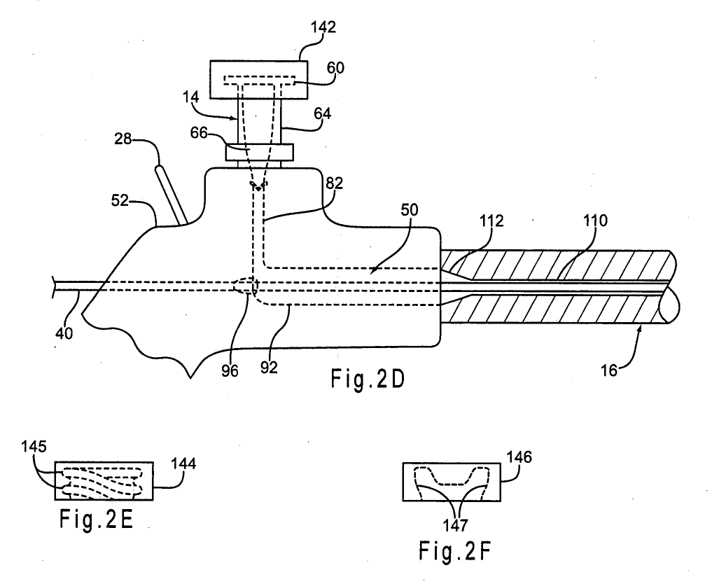

A port 14 is connected to the upper portion 52 of the finger portion 22 of the handle 12. As best seen in FIG. 2A, the port 14 includes a luer connector 60 having an upper rim 62 that is secured to an associated syringe or other infusion device used to infuse a cleaning solution into the passageway 50 formed in the upper portion 52 of the finger portion 22 of the handle 12. The port 14 also includes a collar portion 64. The port 14 defines a port passageway 66 that connects to the passageway 50.

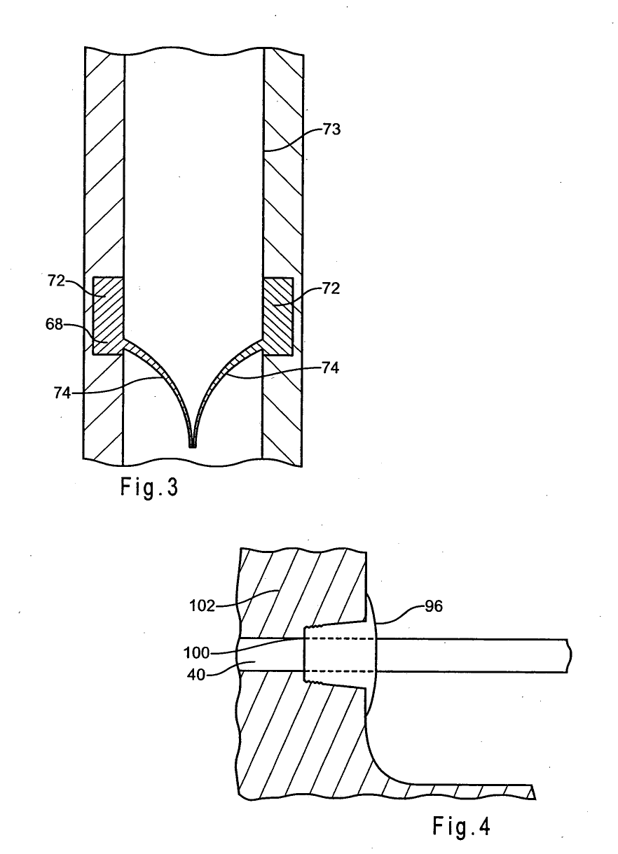

In the illustrated embodiment, a 1-way valve 68 is located in the port 14. The valve 68 can include conventional valves such as the duckbill valve illustrated in FIG. 3. Other conventional valves such as a flapper valve or ball valve may also be implemented with the present invention. The valve 68 includes two side portions 72 mounted to inner wall 73 of the port 14 in the illustrated embodiment. Two flexible portions 74 seal a portion of the port passageway 66 and the passageway 50, but allow for the introduction of a cleaning solution through the port 14. In particular, the valve 68 prevents the escape of C02 through the instrument during a surgical procedure. As a result, the valve 68 helps minimizes the infiltration and collection of bio-burden during the surgical procedure by preventing it from being passed into the instrument with infiltrating C02 . Yet, the port 14 also provides access for conventional cleaning solutions such as enzymatic detergents that may be infused through the port passageway 66 and into the passageway 50 for the cleaning and sterilization processes.

An alternate embodiment for the location of the one-way valve 80 is illustrated in FIG. 2B. The embodiment of FIG. 2 B is essentially the same as the embodiment of FIGS. 1 and 2A with the exception of the location of the valve 80. The valve 80 is located in the upper portion 82 of the passageway 50. The location of the valve 80 has the advantage of being completely contained within the body of the handle, but may create a more complicated manufacturing and assembly process.

Referring again to FIG. 2A, the upper portion 52 of the handle 12 includes a flushing chamber 90 that forms a portion of the passageway 50. In the rear portion 92 of the flushing chamber 90, a seal 96 is provided around the operating rod 40 as it passes out of the flushing chamber 90. With particular reference to FIG. 4, the seal 96 includes a channel 100 through which the operating rod 40 passes. The seal 96 is mounted to a rear portion 102 of the upper portion 52 of the finger portion 22 of the handle 12. In the illustrated embodiment, the seal 96 is threaded into engagement with the rear portion 102. In one embodiment, the seal 96 is formed from stainless steel and is coated with Teflon™. It should be recognized that other known means of attachment such as adhesives or compression fit materials may be implemented. Also, other materials known to those of ordinary skill in the art may be used to form the seal 96. The seal 96 forms an enclosure around the operating rod 40 as it exits the flushing chamber 90 to prevent the escape of C02 during the surgical procedure and the infiltration of bio-burden into the lumen of the instrument. In addition, the seal 96 performs an anti-back flush function that helps prevent cleaning solution passing through the flushing chamber 90 from exiting through the rear portion 102 of the handle 12, thus increasing the pressure of the flush and increasing the flushing efficiency.

The flushing chamber 90, as illustrated in FIG. 2A, has a significantly larger diameter than the diameter of the housing passageway 11O. A throat portion 112 interconnects the flushing chamber 90 and the housing passageway 110. The throat portion 112 has a cornerless and smooth surface with a smoothly or continuously decreasing diameter that creates a “Venturi” type effect for any cleaning solution passing therethrough. In particular, the decrease in volume between the flushing chamber 90 and the somewhat narrow space in the housing passageway 110 causes the cleaning solution passing therethrough to have an increased flow rate and a pressurized laminar flow which is highly effective in removing bio-burden and microorganisms from the instrument during the flushing and cleaning process. This laminar flow design is also very effective in removing residual moisture from the lumen after sterilization thus eliminating the risk of contamination from waterborne pathogens. It is also important to recognize that the diameter of the operating rod 40 is such that it is spaced closely adjacent to the walls that form the housing passageway 110. The relatively close or tight space between these surfaces has a number of advantages such as increasing the cleaning solution flow rate, increasing the flushing effectiveness and decreasing the area in which bio-burden may accumulate. According to one embodiment, the space between the operating rod 40 and walls that form the housing passageway 110 should be between 0.002-0.010 inches.

FIG. 2C illustrates an alternate embodiment that operates in the same general manner as the embodiment of FIG. 2A with the exception of the size and shape of the operating rod 130 and the housing passageway 132. The operating rod 130 has a substantially increased size or diameter relative to the operating rod 40 of FIG. 2A. The housing passageway 132 also has a substantially increased diameter relative to the housing passageway 110 of FIG. 2A. The operating rod 130 includes a throat portion 134 that forms the same “Venturi” type effect for the cleaning solution passing from the flushing chamber 140 and into the housing passageway 132 adjacent the operating rod 130. As in the earlier embodiment, the operating rod 130 should be sized to be closely adjacent the walls that form the housing passageway 132.

FIG. 2D illustrates an alternate embodiment similar to the embodiment of FIG. 2A with the exception of a cap 142. The cap 142 is added to the collar portion 64 such that it covers the luer connector 60. The cap 142 functions similarly to the valve 68 by helping prevent C02 from escaping through the luer connector 60 during a surgical procedure.

FIG. 2E and FIG. 2F are alternate embodiments of the cap 142 in FIG. 2D. Specifically, FIG. 2E is an example of a screw-on cap 144. The screw-on cap 144 is attached over the luer connector 60 by twisting the screw-on cap 144 such that it fastens to the collar portion 64. For example, the screw-on cap 144 may have threads 145 that correspond to threads in the collar portion 64 allowing the screw-on cap 144 to be screwed onto the collar portion 64. In a second alternate embodiment, FIG. 2F shows an example of a snap-on cap 146. The snap-on cap 144 is snapped onto the collar portion 64 over the luer connector 60 such that the grooves 147 in the snap-on cap allow it to fit tightly over the luer connector. It should be recognized that other known cap constructions that provide a leak-proof seal with the luer may be implemented with the present invention.

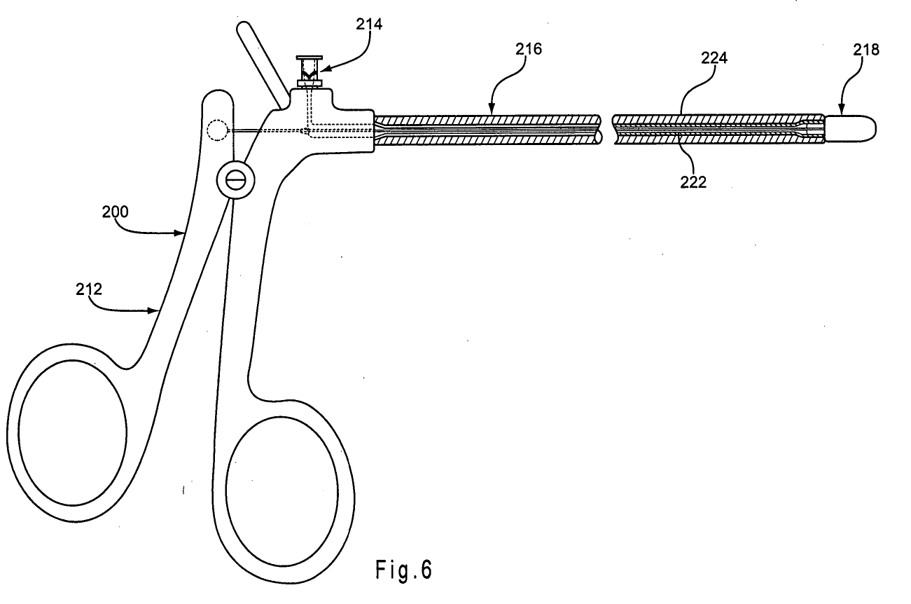

FIG. 5A illustrates the distal end 150 of the housing 16 associated with the embodiment of FIGS. 1 and 2A. The end portion 152 of the operating rod 40 passes through the second throat portion 154 and connects to the tool 18. Again, the throat portion 154 is cornerless and smooth in order to prevent the accumulation of bio-burden. More specifically, as known in the art, the end portion 152 of the operating rod 40 is connected to the tool 18 at the hinge 160. As also recognized in the art, the tool 18 can take a wide variety of forms such as a grasping forcep, a curved dissecting forcep, a curved Maryland dissector, a Babcock grasping forcep and other related tools.

The operating rod 40 can be formed from conventional metals with stainless steel being the preferred material. In the preferred embodiment, the operating rod 40 and the passageway 50 are superfinished to meet an ANSI B46 standard of 2-8 microinches of roughness. The operating rod 40 and passageway can then be plated with materials such as gold, chrome or nickel with gold being the most preferred material. The use of superfinishing and a plating material creates a very smooth surface to which it is more difficult for bio-burden to attach. In addition, such a surface is more readily flushed, cleaned and sterilized.

FIG. 5 B illustrates the distal end 170 of the instrument associated with the embodiment of FIG. 2C and operates in essentially the same manner as the embodiment of FIG. 1 and 2A. The end portion 172 of the operating rod 130 passes through the second throat portion 174 and connects to the tool 18.

FIG. 6 illustrates a further embodiment of a laparoscopic instrument 200 in accordance with the present invention. The laparoscopic instrument 200 is constructed in essentially the same manner as the instrument 10 with exception of the construction of the housing 216. The instrument 200 includes a handle 212, a port 214, an elongated housing 216 and a tool 218. The elongated housing 216 is formed of at least two materials. The inner portion 222 is formed from a stainless steel material and has a relatively thin thickness. The outer portion 224 is formed from a nonconducting material as described with reference to the housing 16 of FIG. 1 and is relatively thick.

The embodiments described above and shown herein are illustrative and not restrictive. The scope of the invention is indicated by the claims rather than by the foregoing description and attached drawings. The invention may be embodied in other specific forms without departing from the spirit of the invention. For example, the type and size of the instrument and portions of the instrument such as the operating rod may be designed in a manner other than as specifically illustrated in the figures. Accordingly, these and any other changes which come within the scope of the claims are intended to be embraced herein.

I claim:

1. A surgical instrument comprising:
a) a handle;
b) an elongated housing connected to a handle, the housing having a proximal end and a distal end, the handle and the housing defining an interior passageway, the elongated housing having a diameter substantially larger than a diameter of the interior passageway;
c) a movable rod located within the passageway and passing through both the first and second throat portions of the housing, d) a tool connected to the rod and extending from the distal end of the elongated housing.

2. The surgical instrument of claim 1 wherein the rod is closely spaced adjacent a wall forming the interior passageway.

3. The surgical instrument of claim 2 wherein the housing is formed from a nonconductive material.

4. The surgical instrument of claim 3 wherein the material forming the housing is nonporous and non-ferrous.

5. The surgical instrument of claim 4 wherein the material forming the housing is a unidirectional graphite carbon-fiber.

6. The surgical instrument of claim 2 further comprising a seal connected to the proximal end of the rod.

7. The surgical instrument of claim 2 further comprising a port connected to the handle.

8. The surgical instrument of claim 7 wherein the port is in fluid communication with the passageway.

9. The surgical instrument of claim 8 wherein the port further comprises a valve.

10. A surgical instrument that is insertable into a body, comprising:
a) a handle;
b) an elongated housing connected to the handle, the housing having a proximal end and a distal end, the housing and the handle defining an interior passageway;
c) a movable rod located within the passageway of the handle and the housing, the rod passing through a rear portion of the handle and through the passageway, a tool connected to a distal end of the rod;
d) a seal connected to the rod and handle adjacent the passageway.

11. The surgical instrument of claim 10 wherein the seal is connected to the rear portion of the handle.

12. The surgical instrument of claim 11 wherein the rod passes through the seal.

13. The surgical instrument of claim 12 wherein the seal is formed from stainless steel.

14. The surgical instrument of claim 13 wherein the seal has a coating.

15. The surgical instrument of claim 14 wherein the coating is Teflon or other non-ferrous, non-conducting material.

16. The surgical instrument of claim 12 wherein the rod is closely spaced adjacent a wall forming the passageway.

17. The surgical instrument of claim 16 wherein the passageway includes a throat portion.

18. The surgical instrument of claim 17 wherein the passageway includes a first and a second throat portions.

19. A surgical instrument that is insertable into a body, comprising:
a) a handle;
b) an elongated housing connected to the handle, the handle and the housing defining an interior fluid passageway;
c) a movable rod located within the fluid passageway of the handle and housing;
d) a port connected to the handle and in fluid communication with the interior fluid passageway; and
e) a valve connected to one of the port and the passageway.

20. The surgical instrument of claim 19 wherein the valve is connected to the port.

21. The surgical instrument of claim 19 wherein the valve is connected to the passageway.

22. The surgical instrument of claim 19 wherein the valve is a one way valve.

23. The surgical instrument of claim 22 wherein the valve is a duck bill valve.

24. The surgical instrument of claim 22 wherein the handle further comprises a flushing chamber forming a portion of the passageway.

25. The surgical instrument of claim 24 wherein the passageway includes a throat portion.

26. The surgical instrument of claim 25 wherein the passageway includes a first and a second throat portions.

27. The surgical instrument of claim 26 further comprising a seal connected to the rod and the handle adjacent the passageway.

28. The surgical instrument of claim 27 further comprising a port connected to a handle.

29. The surgical instrument of claim 28 wherein the port is in fluid communication with the passageway.

30. The surgical instrument of claim 29 wherein the port further comprises a valve.

31. A surgical instrument comprising:
a) a handle;
b) an elongated housing connected to a handle, the housing having a proximal end and a distal end, the handle and the housing defining an interior passageway, the housing being formed from a first nonconductive material;
c) a movable rod located within the passageway and passing through both the first and second throat portions of the housing; and
d) a tool connected to the rod and extending from the distal end of the elongated housing.

32. The surgical instrument of claim 31 wherein the housing is formed from a first material and a second separate material.

33. The surgical instrument of claim 32 wherein the first material is stainless steel.

34. The surgical instrument of claim 33 wherein the second material is a nonporous and non-ferrous material.

35. The surgical instrument of claim 34 wherein the housing is formed from a single material.

36. The surgical instrument of claim 35 wherein the housing is formed from stainless steel.
